(19) United States
(12) Patent Application Publication
(10) Pub. No.: US 2026/0217776 A1
(43) Pub. Date: Jul. 30, 2026

(54) COMPOUND WITH ANTITUMOR ACTIVITY, CORRESPONDING PRODUCTION METHOD AND CORRESPONDING USE

(71) Applicant: Angelo Michele Palmieri, San Nicandro Garganico (fg) (IT)

(72) Inventors: Angelo Michele PALMIERI, San Nicandro Garganico (fg) (IT); Vincenzo DE FILIPPIS, Padova (pd) (IT); Marina ZICHE, Firenze (fi) (IT); Mario PAZZAGLI, Firenze (fi) (IT); Rosa DI LIDDO, Rovigo (ro) (IT); Sandra DONNINI, Monteriggioni (si) (IT); Girolama IADICICCO, Padova (pd) (IT); Concetta Antonia MONTEMURNO, San Nicandro Garganico (fg) (IT); Fiorenzo REFOSCO, Valdagno (vi) (IT)

(21) Appl. No.: 19/158,267

(22) PCT Filed: Feb. 21, 2024
(86) PCT No.: PCT/IT2024/050039

§ 371 Date: Aug. 20, 2025

(30) Foreign Application Priority Data
Feb. 21, 2023 (IT) 102023000002955

(51) Int. Cl. C07K 14/46; A61K 38/00; A61P 35/00

(52) U.S. Cl. CPC C07K 14/46; A61P 35/00; A61K 38/00

(57) ABSTRACT

A compound with antitumor activity comprising a python serum protein and its use in the treatment of cancer are provided.

### FIELD OF THE INVENTION

The present invention concerns a compound with antitumor activity, in particular a compound of natural origin with antitumor activity, a method for producing it as well as a use thereof.

### BACKGROUND OF THE INVENTION

Chemotherapy drugs are classified according to their chemical structure or biological activity (see “The Pharmacological Basis of Therapeutics” by A. Goodman and M. Gilman, Pergamon Press, New York, 2005). In many cases, antitumor drugs act on the cellular life cycle by blocking cell growth.

The classification of chemotherapeutics also includes natural compounds or their derivatives (such as Taxol or vincristine) capable of binding to tubulin and therefore inhibiting mitosis.

Patent application WO 2013/021339 describes proteins having apoptotic and antitelomerase activity on cancer cells, these proteins are useful for the preparation of pharmaceutical formulations having selective apoptotic capacity on cancer cells and cancer stem cells. The proteins known from this document are of natural origin, in particular they come from the African rock python (Python sebae), and have a tetrameric structure composed of four subunits. Their sequences are as follows:
SEQ ID N 1
HKCEICHGFG DDCDGYEEEC PSPEDKCGKI LIDIALAPVS FRATHKNCFS SSICKLGRVD IHVWDGVYMR GRTNCCDNDQ CEDQPLPGLP LSLKNGLYCP GAFGI FTEDS TEHEVKCRGT ETMCLDLVGY REENYAGNIT YNIKGCVSSC PLLTLSERGH EGRKNDLKKV ECREALKPAS SD
SEQ ID N 2
HKCEICHGFG DDCDGYQEEC PSPEDKCGKI LIDIALAPVS FRATHKNCFS SSICKLGRVD IHVWEGVYIR GRTNCCDNDQ CEDQPLPGLP LSLKNGLYCP GAFGI FTEYS TEHEVKCRGT ETMCLDLVGY REENYVGNIT YNIKGCVSSC PLVTLSERGH EGRKNDLKKV ECREALKYES SD
SEQ ID 3
HKCEICHGFG DDCDGYEEEC PSPEDQCGKI LIDIALAPLS FRATHKNCFS SSICKLGRVD IHVWDGVYMR GRTNCCDNDQ CEDEPLPGLP LSLKNGLYCP GAFGIFTEDS TEHEVKCRGT ETMCLDLVGY REENYAGNIT YNIKGCVSSC PLITLSERGH EGRKNDLKKV ECREALKPAS SD
SEQ ID N 4
HKCEICHGFG DDCDGYEEEC PSPEDQCGKI LIDIALAPLS FRATHKNCFS SSICKLGRVD IHVWEGVYIR GKTNCCDNDQ CEDEPLPGLP LSLKNGLYCP GAFGIFTEYS TEHEVKCRGT ETMCLDLVGY REENYVGNIT YNIKGCVSSC PLITLSERGH EGRKNDLKKV ECREALKYES SD

One disadvantage of known proteins lies in the fact that, despite their selective antitumor activity, they are problematic for the development of pharmaceutical products. These problems are due to their oligomeric structure and their high molecular weight, greater than 20 kDa.

Another disadvantage of known proteins is that, since they are of natural origin, the only way to obtain them is by taking them from their natural source, in this case a specific python from the African continent. They are therefore subject to poor source availability, which is particularly problematic for pharmaceutical type industrial production.

Yet another disadvantage of known proteins is that they are difficult to modify, which leads to problems in possibly optimizing a pharmaceutical product based on such proteins.

There is therefore the need to perfect a compound with antitumor activity that can overcome at least one of the disadvantages of the state of the art.

To do this, it is necessary to solve the technical problem of providing a compound with antitumor activity that is suitable for the development of pharmaceutical products.

In particular, one purpose of the present invention is to provide a compound with antitumor activity which has a lower molecular weight and a simpler structure than known proteins.

Another purpose of the present invention is to provide a compound with antitumor activity which does not have availability problems.

Another purpose of the present invention is to provide a compound with antitumor activity that can be modified, so as to allow the optimization of any candidate pharmaceutical products.

Another purpose of the present invention is to provide a compound with antitumor activity that has a natural origin.

The Applicant has devised, tested and embodied the present invention to overcome the shortcomings of the state of the art and to obtain these and other purposes and advantages.

### SUMMARY OF THE INVENTION

The present invention is set forth and characterized in the independent claims. The dependent claims describe other characteristics of the present invention or variants to the main inventive idea.

In accordance with the above purposes and to resolve the technical problem disclosed above in a new and original way, also achieving considerable advantages compared to the state of the prior art, a compound according to the present invention with antitumor activity comprises a peptide isolated from one of the proteins of the state of the art with sequence SEQ ID N 1, SEQ ID N 2, SEQ ID N 3 or SEQ ID N 4.

The peptide has the sequence:
SEQ ID N 5
¹⁴⁹SCPLLTLSERGHEGRKNDLKKVECR¹⁷³
or SEQ ID N 6
¹⁴⁹SCPLVTLSERGHEGRKNDLKKVECR¹⁷³
or SEQ ID N 7
¹⁴⁹SCPLITLSERGHEGRKNDLKKVECR¹⁷³

In particular, the peptide corresponds to the region 149-173 of one of the above-mentioned sequences, more in particular SEQ ID N 5 corresponds to the region 149-176 of SEQ ID N 1, SEQ ID N 6 corresponds to the region 149-173 of SEQ ID N 2 and SEQ ID N 7 corresponds to the region 149-173 of SEQ ID N 3 or SEQ ID N 4.

Preferably, the peptide corresponds to the region 149-173 of sequence SEQ ID N 1, that is, it has the sequence SEQ ID N 5.

Doing so achieves at least the advantage of having a compound with antitumor activity which, in addition to being of natural origin like the protein from which it was isolated, has a much simpler structure and a much lower molecular weight (approximately 2.9 kDa) compared to known proteins. This makes this compound more suitable for a pharmaceutical-type development.

Furthermore, the compound according to the present invention can be obtained synthetically, in the laboratory, which solves the problem of poor availability of the source. Since the compound can be synthesized, it is clear that it can also be easily modified, which allows it to be optimized with a view of producing pharmaceutical products.

In accordance with another aspect of the present invention, a method for producing the compound with antitumor activity provides a peptide synthesis, preferably in the solid phase. Advantageously, the method provides to use the compound Fmoc for the protection of the α-amino group of the amino acids of the peptide during the synthesis.

In accordance with another aspect of the present invention, the compound with antitumor activity disclosed above is provided for use in the treatment of cancer cells and cancer stem cells. Advantageously, the cancer cells are human epidermoid carcinomas.

In accordance with another aspect of the present invention, it is provided to use the compound with antitumor activity disclosed above for the preparation of pharmaceutical formulations having selective antitumor activity on cancer cells and on cancer stem cells. Advantageously, the cancer cells are human epidermoid carcinomas.

### DESCRIPTION OF THE DRAWINGS

These and other aspects, characteristics and advantages of the present invention will become apparent from the following description of an embodiment, given as a non-restrictive example with reference to the attached drawings wherein:

FIG. 1 is a spectrum obtained by means of tandem mass spectrometry of a compound with antitumor activity, according to the present invention;

FIG. 2 is a chromatogram obtained by means of RP-HPLC of the compound with antitumor activity, according to the present invention;

FIG. 3 is a deconvoluted mass spectrum of the compound with antitumor activity, according to the present invention;

FIGS. 4A and 4B show the results of a cytotoxic activity assay of the compound with antitumor activity according to the present invention, and show the viability of three cancer cell lines treated and not treated with the compound after 1 day and 6 days, respectively; and

FIGS. 5A and 5B show the results of an apoptotic activity assay of the compound with antitumor activity according to the present invention, and show the apoptosis of three cancer cell lines treated and not treated with the compound after 1 day and 6 days, respectively.

We must clarify that the phraseology and terminology used in the present description, as well as the figures in the attached drawings also in relation as to how described, have the sole function of better illustrating and explaining the present invention, their purpose being to provide a non-limiting example of the invention itself, since the scope of protection is defined by the claims.

To facilitate comprehension, the same reference numbers have been used, where possible, to identify identical common elements in the drawings. It is understood that elements and characteristics of one embodiment can be conveniently combined or incorporated into other embodiments without further clarifications.

### DESCRIPTION OF SOME EMBODIMENTS OF THE PRESENT INVENTION

Unless otherwise defined, all the technical and scientific terms used here and hereafter have the same meaning as commonly understood by a person with ordinary experience in the field of the art to which the present invention belongs. Even if methods and materials similar or equivalent to those described here can be used in practice and in the trials of the present invention, the methods and materials are described hereafter as an example. In the event of conflict, the present application shall prevail, including its definitions. The materials, methods and examples have a purely illustrative purpose and shall not be understood restrictively.

All measurements are carried out at 25° C. (ambient temperature) and at atmospheric pressure, unless otherwise indicated. All temperatures are in degrees Celsius, unless otherwise indicated.

All percentages and ratios indicated shall be understood to refer to the weight of the total composition (w/w), unless otherwise indicated.

All percentage ranges indicated here are given with the provision that the sum with respect the overall composition is 100%, unless otherwise indicated.

All the intervals reported here shall be understood to include the extremes, including those that report an interval “between” two values, unless otherwise indicated.

The present description also includes the intervals that derive from uniting or overlapping two or more intervals described, unless otherwise indicated.

The present description also includes the intervals that can derive from the combination of two or more punctual values, unless otherwise indicated.

The following abbreviations are used in the present description: SPPS, solid phase peptide synthesis; Fmoc, fluorenylmethyloxycarbonyl; NMP, N-Methylpyrrolidone; DIEA, diisopropylethylamine; DMF, dimethylformamide; HOBt, hydroxybenzotriazole; Pbf, pentamethyldihydrobenzofuran-5-sulfonyl; TFA, trifluoroacetic acid; EDT, ethanedithiol; TIPS, triisopropylsilane; RP-HPLC, reversed phase high performance liquid chromatography; ACN, acetonitrile; Da, dalton; TRIS, tris(hydroxymethyl)aminomethane; β-ME, β-mercaptoethanol.

As a compound according to the present invention we have identified, in the sequence SEQ ID N 1 of the corresponding known protein, a peptide with the sequence:
SEQ ID N 5
¹⁴⁹SCPLLTLSERGHEGRKNDLKKVECR¹⁷³

which maintains antitumor activity, similarly to what is observed with the original protein. The peptide sequence corresponds to the sequence between positions 149 and 173 of the sequence SEQ ID N 1.

Even if the peptide is of natural origin, and therefore obtainable from a natural source, a method for its production provides a peptide synthesis.

In particular, a solid phase peptide synthesis (SPPS) was performed using a PS3 peptide synthesizer from Protein Technologies (Tucson, AZ), exploiting the Fmoc chemistry for deprotection of the terminals during the synthesis. As a support, a resin derivatized with the C-terminal amino acid of the peptide to be synthesized was used. The following reagents were used for the synthesis:

| | |
|---|---|
| Solvent | N-methylpyrrolidone (NMP) |
| Activation | 0.35M di-isopropylethylamine (DIEA) in |
| solution | N,N′-dimethylformamide (DMF) |
| (—COOH) | |
| Deprotection | 20% piperidine in NMP |
| solution (Fmoc) | |
| Activators | N1-hydroxybenzotriazole(HOBt)/hydroxy |
| | benzotriazolyluronium hexafluorophosphate |
| | (HBTU) in equimolar ratios |
| Resin | Fmoc-Arg(Pbf)-Wang resin |
| Unblocking | 92% TFA (synthesis grade), 3% H₂O |
| mixture | milliQ, 3% EDT, 2% TIPS |

After drying the crude peptide in a nitrogen stream, it was dissolved in H₂O-TFA (0.1% v/v) and analyzed using RP-HPLC.

For the RP-HPLC characterization, an analytical column of the Vydac 218TP54 C18 type (4.6×250 mm; 5 μm, 300 Å) was used, equilibrated with a 10% acetonitrile solution in water, in the presence of TFA 0.1%. For the elution, a gradient of mobile phase B (acetonitrile+0.078% TFA) from 10 to 30% in 20 minutes was used, at a flow rate of 0.8 ml/min. The absorbance of the eluate was recorded at a wavelength of 226 nm.

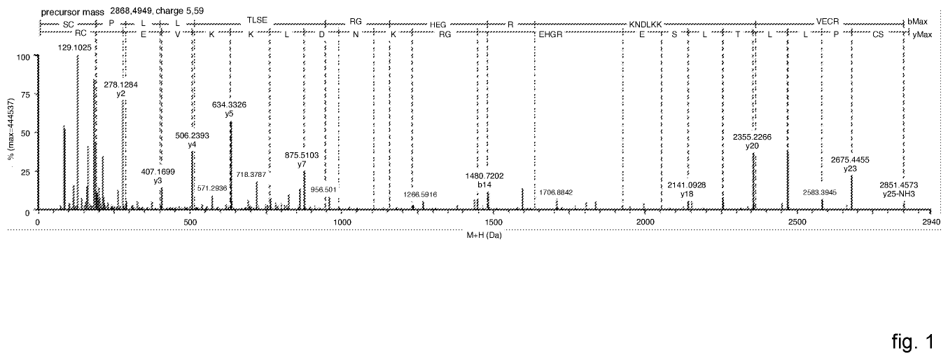

The material eluted in correspondence with the chromatographic peak with the greatest intensity (approximately 32% acetonitrile) was freeze-dried using a SpeedVac concentrator and subjected to chemical characterization by means of high-resolution mass spectrometry, using the Xevo-G2S Q-TOF spectrometer (Waters, Milford, MO) equipped with an electrospray ionization source. The experimental mass obtained from the analysis of the peptide is in agreement with the theoretical mono-isotopic molecular weight of the amino acid sequence (2867.49 Da). The correct peptide sequence was also confirmed using tandem mass spectrometry (FIG. 1).

Once the correct synthesis was confirmed, the peptide obtained was subjected to an oxidative renaturation process in order to promote the formation of the disulfide bridge between Cys150 and Cys172, present in the sequence of the natural protein. The mixture used for the cysteine oxidation reaction, which required 16 hours and was monitored by means of RP-HPLC, was composed as follows: 100 mM TRIS·HCl pH 8.3, 250 UM β-ME, peptide concentration 0.5 mg/mL.

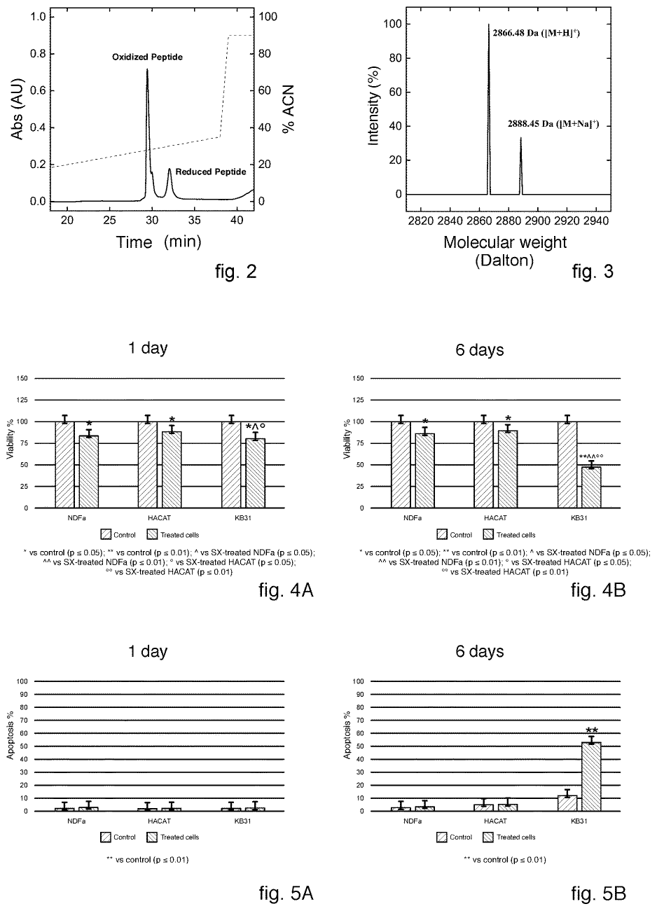

Once the oxidation process was completed, the reaction mixture was acidified by adding TFA and analyzed by means of RP-HPLC using the same conditions previously reported (FIG. 2). The material eluted in correspondence with the chromatographic peak which formed following the oxidation reaction (approximately 28% acetonitrile) was subjected to freeze-drying and analyzed using high-resolution mass spectrometry (FIG. 3).

The experimental mass value, obtained from the analysis of the oxidized peptide, is in agreement with the theoretical mono-isotopic molecular weight expected for the amino acid sequence in which the two cysteines present are paired (2865.49 Da).

The peptide thus synthesized and characterized was then used to perform cytotoxic activity and apoptotic activity assays.

The cytotoxic activity assays were performed using a KD31 human epidermoid carcinoma cell line (American Type Culture Collection) and, as control, non-tumor cell lines of normal dermal fibroblasts (NDFa) and immortalized human keratinocytes (HACAT).

The used cells were placed in culture dishes (Corning®) and treated with the peptide (at a concentration of 150 μM) for 1 day and 6 days. In parallel, the same lines were prepared, without being treated with the peptide, as a negative control.

The analyzes were performed with a resazurin-based cytotoxicity assay (Sigma Aldrich) and a trypan blue exclusion assay. The results, shown in FIGS. 4A and 4B, are expressed as average values in percentages considering a standard error of the mean (SEM).

The same cell lines were used to verify the apoptotic activity of the peptide, providing the same treatment and the same negative controls. The percentage of apoptotic cells was determined using a BD™ cell viability kit (BD Biosciences), following the manufacturer's instructions. The results are expressed as average values in percentages taking into account the standard error of the mean (SEM).

After 1 day of treatment with the peptides, a decrease in cell viability was observed in all three cell lines tested, already with a greater decrease observed for the KB31 line compared to the other two NDFa and HACAT lines. After 6 days of treatment, the viability of the NDFa and HACAT lines remained at approximately the same level observed after 1 day (approximately 80% and 85%, respectively) while the viability of the KB31 line decreased from 77% after 1 day, to approximately 45% after 6 days. The viability of untreated cells remained at 100% for all three cell lines.

Similar results were obtained in the apoptotic activity assay. After 1 day, less than 5% apoptosis was observed for all cell lines. After 6 days, apoptosis measured in non-tumor NDFa and HACAT cells remained less than 5%, while apoptosis detected for the KB31 tumor line treated with the peptide rose to more than 50%. Negative controls remained less than 5% for the non-tumor lines, and rose to just over 10% for the KB31 tumor line.

These assays show that the peptide according to the invention has good selectivity toward tumor cells, in particular human epidermal KB31 cells.

It is clear that modifications and/or additions of parts may be made to the compound with antitumor activity, to the corresponding production method and use as described heretofore, without departing from the field and scope of the present invention, as defined by the claims.

It is also clear that, although the present invention has been described with reference to some specific examples, a person of skill in the art will be able to create other equivalent forms of compound with antitumor activity, corresponding production method and corresponding use, having the characteristics as set forth in the claims and hence all coming within the field of protection defined thereby.

In the following claims, the sole purpose of the references in brackets is to facilitate their reading and they must not be considered as restrictive factors with regard to the field of protection defined by the claims.

1. A compound with antitumor activity comprising a peptide having a sequence selected from the group consisting of:
SEQ ID NO: 5
SCPLLTLSERGHEGRKNDLKKVECR
SEQ ID NO: 6
SCPLVTLSERGHEGRKNDLKKVECR
and
SEQ ID NO: 7
SCPLITLSERGHEGRKNDLKKVECR.

2. The compound of in claim 1, wherein the peptide has the sequence of SEQ ID N 5.

3. A method for producing a compound with antitumor activity as in claim 1, comprising a peptide synthesis step.

4. The method of claim 3, wherein the peptide synthesis step is carried out in the solid phase.

5. The method of claim 3, wherein the method comprises using the compound Fmoc for the protection of amino acids during the synthesis.

6. A method for treating cancer cells and cancer stem cells comprising the use of the compound with antitumor activity of claim 1.

7. The method of claim 6, wherein the cancer cells are human epidermoid carcinoma cells.


SEQUENCE LISTING (7 sequences)

SEQ ID NO: 1 - protein, 182 aa, Python sebae
HKCEICHGFGDDCDGYEEECPSPEDKCGKILIDIALAPVSFRATHKNCFSSSICKLGRVDIHVWDGVYMRGRTNCCDNDQCEDQPLPGLPLSLKNGLYCPGAFGIFTEDSTEHEVKCRGTETMCLDLVGYREENYAGNITYNIKGCVSSCPLLTLSERGHEGRKNDLKKVECREALKPASSD

SEQ ID NO: 2 - protein, 182 aa, Python sebae
HKCEICHGFGDDCDGYQEECPSPEDKCGKILIDIALAPVSFRATHKNCFSSSICKLGRVDIHVWEGVYIRGRTNCCDNDQCEDQPLPGLPLSLKNGLYCPGAFGIFTEYSTEHEVKCRGTETMCLDLVGYREENYVGNITYNIKGCVSSCPLVTLSERGHEGRKNDLKKVECREALKYESSD

SEQ ID NO: 3 - protein, 182 aa, Python sebae
HKCEICHGFGDDCDGYEEECPSPEDQCGKILIDIALAPLSFRATHKNCFSSSICKLGRVDIHVWDGVYMRGRTNCCDNDQCEDEPLPGLPLSLKNGLYCPGAFGIFTEDSTEHEVKCRGTETMCLDLVGYREENYAGNITYNIKGCVSSCPLITLSERGHEGRKNDLKKVECREALKPASSD

SEQ ID NO: 4 - protein, 182 aa, Python sebae
HKCEICHGFGDDCDGYEEECPSPEDQCGKILIDIALAPLSFRATHKNCFSSSICKLGRVDIHVWEGVYIRGKTNCCDNDQCEDEPLPGLPLSLKNGLYCPGAFGIFTEYSTEHEVKCRGTETMCLDLVGYREENYVGNITYNIKGCVSSCPLITLSERGHEGRKNDLKKVECREALKYESSD

SEQ ID NO: 5 - protein, 25 aa, synthetic construct
SCPLLTLSERGHEGRKNDLKKVECR

SEQ ID NO: 6 - protein, 25 aa, synthetic construct
SCPLVTLSERGHEGRKNDLKKVECR

SEQ ID NO: 7 - protein, 25 aa, synthetic construct
SCPLITLSERGHEGRKNDLKKVECR